(19) United States
(12) Patent Application Publication
(10) Pub. No.: US 2009/0298947 A1
(43) Pub. Date: Dec. 3, 2009

(54) POLYMORPHIC AND AMORPHOUS FORMS OF LACOSAMIDE AND AMORPHOUS COMPOSITIONS

(75) Inventors: Tina MUNDORFER, Zagreb (HR); Marina MARKOVIC, Zagreb (HR); Nada KOSUTIC HULITA, Zagreb (HR); Miroslav ZEGARAC, Zagreb (HR)

Correspondence Address: MERCHANT & GOULD PC, P.O. BOX 2903, MINNEAPOLIS, MN, 55402-0903, US

(73) Assignee: PLIVA HRVATSKA D.O.O., Zagreb (HR)

(21) Appl. No.: 12/472,968

(22) Filed: May 27, 2009

Related U.S. Application Data
(60) Provisional application No. 61/056,612, filed on May 28, 2008.

(51) Int. Cl. A61K 31/165; C07C 235/02; A61P 25/00

(52) U.S. Cl. 514/616; 564/158

(57) ABSTRACT

The present invention relates to polymorphic and amorphous forms of Lacosamide, processes of preparing the polymorphic and amorphous forms, pharmaceutical compositions containing the same, therapeutic uses thereof and methods of treatment employing the same.

### CROSS-REFERENCE TO RELATED APPLICATIONS

This application claims the benefit of U.S. Provisional Patent Application Ser. No. 61/056,612, filed May 28, 2008, which is incorporated herein by reference.

### FIELD OF THE INVENTION

The present invention is concerned with new polymorphic and amorphous forms of Lacosamide and amorphous composition containing Lacosamide combined with a pharmaceutical acceptable ingredient, processes for preparing thereof, pharmaceutical compositions containing the same, therapeutic uses thereof and methods of treatment employing the same.

### BACKGROUND

(R)-N-benzyl-2-acetamido-3-methoxypropionamide, known as Lacosamide, has the following structure:

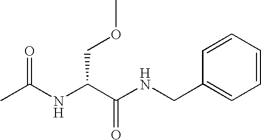

Lacosamide is an anticonvulsant drug useful in the treatment of central nervous system disorders such as epilepsy. This drug is also useful in the treatment of pain, particularly neuropathic pain such as diabetic neuropathic pain. Lacosamide is marketed under the trade name Vimpat® by UCB. It was approved by the FDA as an adjunctive therapy for partial-onset seizures in October 2008.

A number of syntheses of Lacosamide have been reported in U.S. Pat. Nos. RE 38,551; 6,048,899 and 2008/0027137.

Polymorphism, the occurrence of different crystal forms, is a property of some molecules and molecular complexes. A single molecule may give rise to a variety of polymorphs having distinct crystal structures and physical properties like melting point, x-ray diffraction pattern, infrared absorption fingerprint, and solid state NMR spectrum. One polymorph may give rise to thermal behavior different from that of another polymorph. Thermal behavior can be measured in the laboratory by such techniques as capillary melting point, thermogravimetric analysis (“TGA”), and differential scanning calorimetry (“DSC”), which have been used to distinguish polymorphic forms.

The difference in the physical properties of different polymorphs results from the orientation and intermolecular interactions of adjacent molecules or complexes in the bulk solid. Accordingly, polymorphs are distinct solids sharing the same molecular formula yet having distinct advantageous physical properties compared to other polymorphs of the same composition or complex.

One of the most important physical properties of pharmaceutical compositions is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. Different polymorphs or polymorphs of the same pharmaceutical compositions can and reportedly do have different aqueous solubilities.

The discovery of new polymorphic forms and solvates of a pharmaceutically useful composition provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. Therefore, there is a need for additional polymorphs of Lacosamide.

### SUMMARY OF THE INVENTION

In one embodiment, the present invention encompasses crystalline Lacosamide characterized by data selected from a group consisting of a PXRD pattern having peaks at about 8.3, 13.0, 16.6, 17.7 and 21.4 deg±0.2 degrees 2-theta, a PXRD pattern as depicted in FIG. 1 and combination thereof.

In another embodiment, the present invention encompasses a process for the preparation of crystalline Lacosamide characterized by a PXRD pattern having peaks at about 8.3, 13.0, 16.6, 17.7 and 21.4 deg±0.2 degrees 2-theta comprising providing a solution of Lacosamide in ethyl acetate and adding the obtained solution to n-heptane to obtain a suspension comprising the said crystalline form.

In yet another embodiment, the present invention encompasses a process for the preparation of crystalline Lacosamide characterized by a PXRD pattern having peaks at about 8.3, 13.0, 16.6, 17.7 and 21.4 deg±0.2 degrees 2-theta comprising providing a suspension of Lacosamide in ethyl acetate and maintaining the obtained suspension for at least 4 hours, at a temperature of about 40° C. to about 50° C.

In one embodiment, the present invention encompasses crystalline Lacosamide characterized by data selected from a group consisting of a PXRD pattern having peaks at about 5.2, 6.7, 12.6, 16.2, 20.0 and 20.3 deg±0.2 degrees 2-theta, a PXRD pattern as depicted in FIG. 5 and combination thereof.

In another embodiment, the present invention encompasses a process for the preparation of crystalline Lacosamide characterized by a PXRD pattern having peaks at about 5.2, 6.7, 12.6, 16.2, 20.0 and 20.3 deg±0.2 degrees 2-theta comprising providing a solution of Lacosamide in ethyl acetate and adding n-heptane to the obtained solution to obtain a suspension comprising the said crystalline form.

In yet another embodiment, the present invention encompasses a process for the preparation of crystalline Lacosamide characterized by a PXRD pattern having peaks at about 5.2, 6.7, 12.6, 16.2, 20.0 and 20.3 deg±0.2 degrees 2-theta comprising providing a solution of Lacosamide with ethyl acetate and cooling the solution to room temperature to obtain a suspension comprising the said crystalline form.

In another embodiment, the present invention encompasses amorphous Lacosamide.

In one embodiment, the present invention provides pharmaceutical compositions comprising at least one of the above polymorphic and amorphous forms of Lacosamide and pharmaceutically acceptable excipient for use.

In another embodiment, the present invention also encompasses a pharmaceutical composition comprising at least one of the above described polymorphic and amorphous forms of Lacosamide prepared according to the processes of the present invention, and at least one pharmaceutically acceptable excipient.

In another embodiment, the invention encompasses a process for preparing a pharmaceutical composition comprising at least one of the above-described polymorphic and amorphous forms of Lacosamide, and at least one pharmaceutically acceptable excipient.

In another embodiment, the invention encompasses the use of at least one of the above described polymorphic and amorphous form of Lacosamide for the manufacture of a medicament for the treatment of e.g., as an anticonvulsant or for relieving pain.

In yet another aspect, the present invention encompasses a method of treating central nervous system disorders and alleviating pain comprising administering to a subject in need thereof of a pharmaceutical composition comprising at least one of the above-described polymorphic and amorphous forms of Lacosamide, and at least one pharmaceutically acceptable excipient.

In another embodiment, the present invention encompasses an amorphous composition containing Lacosamide combined with a pharmaceutical acceptable ingredient, wherein the pharmaceutical acceptable ingredient is selected from a group consisting of Hypromellose or Hydroxypropyl cellulose.

### BRIEF DESCRIPTION OF THE DRAWINGS

FIG. 1 depicts an X-ray powder diffraction pattern of Form I of Lacosamide.

FIG. 2 depicts an infrared (IR) spectrum of Form I of Lacosamide.

FIG. 3 depicts a DSC thermogram of Form I of Lacosamide (heating rate, 10° C./min).

FIG. 4 depicts a thermogravimetric analysis (TGA) thermogram of Form I of Lacosamide wherein the heating rate is 10° C./min.

FIG. 5 depicts an X-ray powder diffraction pattern of Form II of Lacosamide.

FIG. 6 depicts an IR spectrum of Form II Lacosamide.

FIG. 7. depicts a DSC thermogram of Form II of Lacosamide (heating rate, 10° C./min).

FIG. 8 depicts a TGA thermogram (heating rate, 10° C./min) of Form II of Lacosamide.

FIG. 9 depicts an X-ray powder diffraction pattern of amorphous Lacosamide.

FIG. 10 depicts a DSC thermogram (heating rate, 10° C./min) of amorphous Lacosamide.

FIG. 11 depicts a TGA thermogram (heating rate, 10° C./min) of amorphous Lacosamide.

FIG. 12 depicts an X-ray powder diffraction pattern of an amorphous composition containing Lacosamide combined with Hypromellose.

FIG. 13 depicts an X-ray powder diffraction pattern of Form III of Lacosamide.

### DETAILED DESCRIPTION

The present invention discloses new polymorphic and amorphous forms of Lacosamide and amorphous composition containing Lacosamide combined with a pharmaceutical acceptable ingredient, processes for preparing thereof, pharmaceutical compositions containing the same, therapeutic uses thereof and methods of treatment employing the same.

As used herein the term “room temperature” refers to a temperature of about 20° C. to about 25° C.

As used herein the term “overnight” refers to a period of about 12 to about 18 hours, preferably for about 14 hours.

In one embodiment, the present invention is directed to new polymorphic and amorphous forms of Lacosamide. Thus, in one embodiment, the present invention provides crystalline Lacosamide of form I (the form I polymorph). The form I polymorph of Lacosamide exhibits an X-ray powder diffraction pattern comprising one or more characteristic peaks at about 8.3, 13.0, 16.6, 17.7, 21.4, 24.9, or 25.4 deg±0.2 degrees 2-theta. For example, the form I polymorph of Lacosamide may exhibit two or more, three or more, four or more, five or more, six or more or seven characteristic X-ray powder diffraction peaks at about 8.3, 13.0, 16.6, 17.7, 21.4, 24.9, or 25.4 deg±0.2 degrees 2-theta.

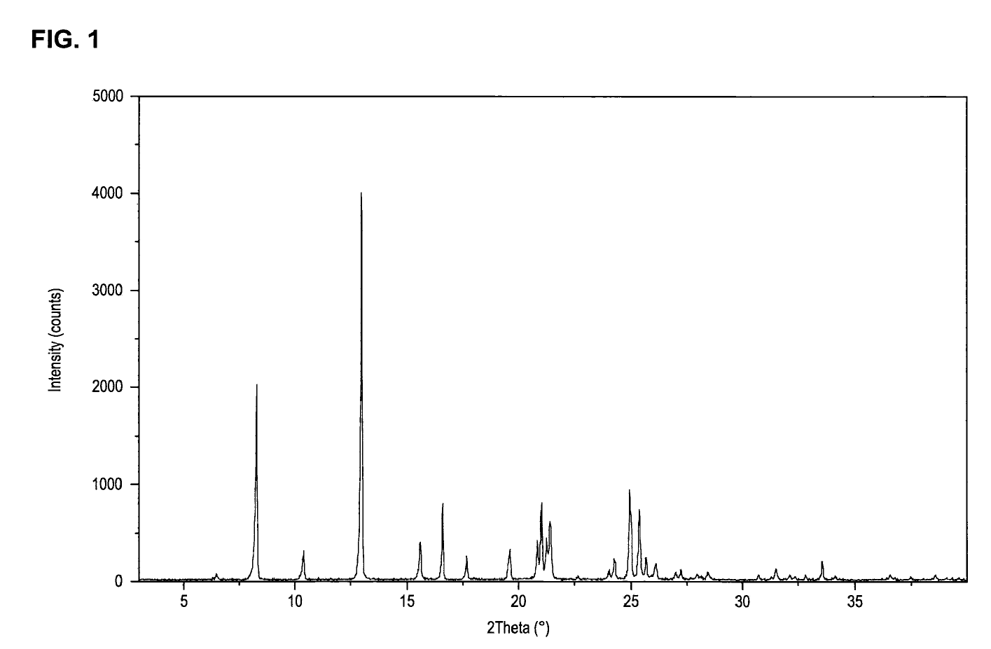

In a preferred embodiment, the present invention encompasses crystalline form I of Lacosamide characterized by data selected from a group consisting of a PXRD pattern having peaks at about 8.3, 13.0, 16.6, 17.7 and 21.4 deg±0.2 degrees 2-theta, a PXRD pattern as depicted in FIG. 1 and combination thereof.

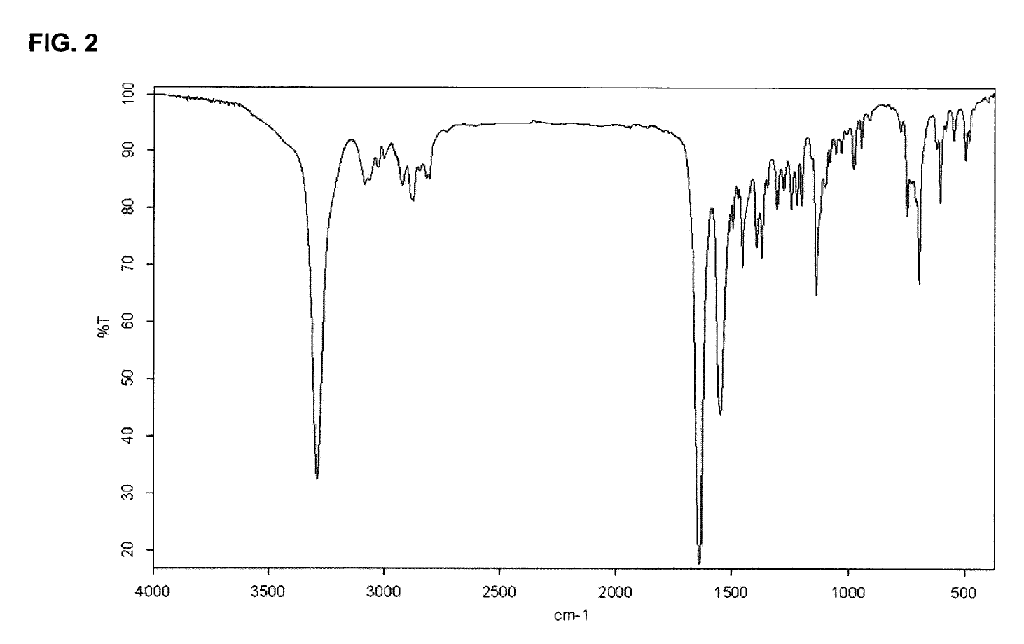

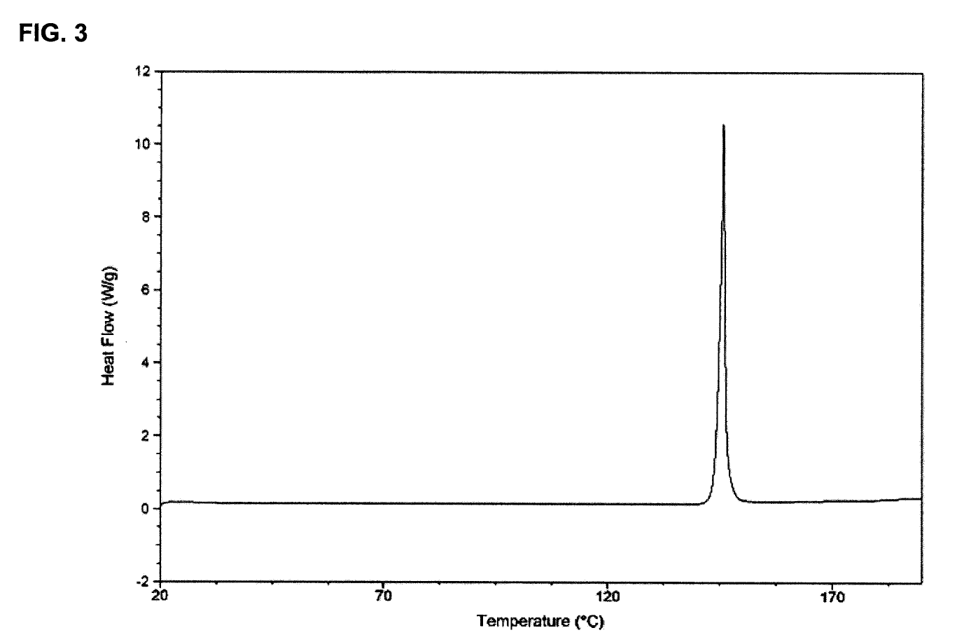

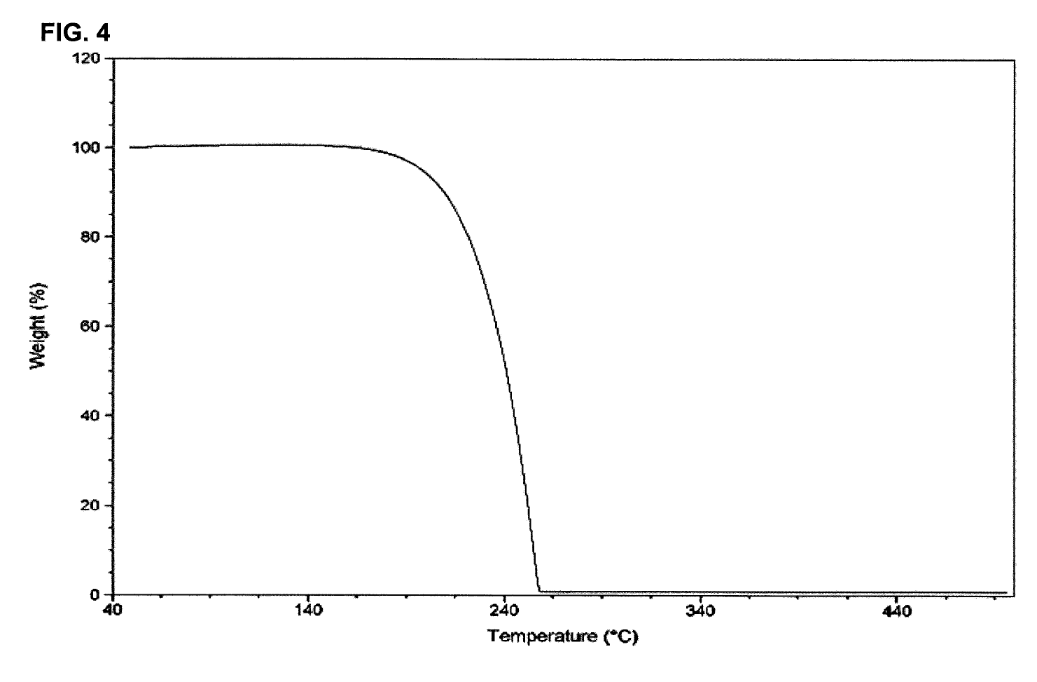

Crystalline form I can be further characterized by data selected from a group consisting of: a PXRD pattern having peaks at about 24.9 and 25.4 deg±0.2 degrees 2-theta, an IR spectrum having absorbance peaks at about 3291, 3088, 2925, 2877, 2820, 1639, 1548, 1455, 1396, 1371, 1139, and 695 cm⁻¹, an IR spectrum as depicted in FIG. 2, a DSC thermogram as shown in FIG. 3 and having peak at about 146° C., and a thermal curve as measured by TGA as shown in FIG. 4.

In a more preferred embodiment, the above form I is polymorphically pure.

As used herein the term polymorphically pure form I corresponds to composition containing Lacosamide form I and not more than about 10% by weight, preferably, not more than 5%, and more preferably, not more than 1% by weight, of Lacosamide characterized by a PXRD pattern having peaks at about 5.2, 6.7, 12.6, 16.2, 20.0 and 20.3±0.2 degrees 2-theta, designated form II of Lacosamide.

The amount of lacosamide form I and form II in the said composition can be measured by solid-state ¹³C NMR or PXRD. For example, the amount of form I can be measured by solid-state 13C NMR using the peak at 137.8 ppm±0.2; and the amount of form II can be measured by any one of the peaks at 5.2 and 16.2 degree±0.2 degrees 2-theta.

The above Lacosamide form I can be prepared by a process comprising providing a solution of Lacosamide in ethyl acetate and adding the obtained solution to n-heptane to obtain a suspension comprising the said crystalline form.

The starting Lacosamide can be prepared for example according to the process reported in U.S. Pat. No. 6,048,899.

Typically, the said solution is obtained by combining Lacosamide and ethyl acetate and heating the said combination. Preferably, heating is done to a temperature of about 60° C. to about 70° C., more preferably, to about 70° C. Preferably, the obtained solution is added to n-heptane at a temperature of about room temperature to about 50° C.

Preferably, the obtained suspension is further cooled to a temperature of about room temperature.

Preferably, the obtained suspension is further maintained. Preferably, the suspension is maintained for a period of about 0.5 to about 4 hours, more preferably, of about 0.5 to about 1 hour, most preferably, for about 0.5 hours.

The above Lacosamide form I can be also prepared by a process comprising providing a suspension of Lacosamide in ethyl acetate and maintaining the obtained suspension for at least about 4 hours at a temperature of about 40° C. to about 50° C.

Preferably, the said suspension is obtained, by combining Lacosamide and ethyl acetate, heating the said combination to a temperature of about 60° C. to about 70° C. to obtain a solution and cooling the obtained solution to a temperature of about 40° C. to about 50° C. to obtain the said suspension comprising the said crystalline form.

Preferably, heating is done at a temperature of about 70° C.

Preferably, the said suspension is maintained for a period of about 4 to about 8 hours, more preferably, of about 4 to about 6 hours, most preferably, for about 4 hours.

The above processes for preparing crystalline Lacosamide form I may further comprises recovery of the said crystalline form from the suspension. The recovery may be done, for example, by filtering the suspension comprising lacosamide form I, washing and drying.

Preferably, washing is done with ethyl acetate or a mixture of ethyl acetate and n-heptane. Preferably, drying is done at a temperature of about 50° C. Preferably, drying is done under vacuum.

The above form I of Lacosamide can be also prepared by a process comprising crystallizing form I Lacosamide from a solution comprising Lacosamide and a solvent selected from a group consisting of: dichloromethane, methyl acetate, ethyl ether, acetone, acetonitrile, chloroform, isopropanol, methanol, ethanol, or mixture of two or more thereof.

In other embodiment, the solution consists of Lacosamide and a solvent selected from a group consisting of: dichloromethane, methyl acetate, ethyl ether, acetone, acetonitrile, chloroform, isopropanol, methanol, ethanol, or mixture of two or more thereof.

Typically, the crystallization comprises providing a solution of Lacosamide in one or a mixture of two or more of the above solvents and cooling the said solution to obtain a suspension comprising the said crystalline form.

The solution is provided by combining Lacosamide with one or a mixture of two or more of the above solvents and heating the said combination. Preferably, heating is done to a temperature of about 40° C. to about 70° C., more preferably, of about 50° to about 60° C.

Preferably, the cooling is to a temperature of about room temperature to about 5° C.

In a preferred embodiment, the cooling is gradual, i.e. first step is cooling to about room temperature, and maintaining for a period of about 3 hours, second step is cooling to about 5° C. and maintaining for a period of about 2 hours.

The process for preparing crystalline lacosamide form I may further comprises recovery of the said crystalline form from the suspension. The recovery may be done, for example, by filtering the suspension comprising lacosamide form I.

In another embodiment, the present invention provides crystalline Lacosamide form II (the form II polymorph). The form II polymorph of Lacosamide may exhibit an X-ray powder diffraction pattern comprising one or more characteristic peaks at about 5.2, 6.7, 12.6, 16.2, 20.0, 20.3, or 23.3 deg±0.2 degrees 2-theta. For example, the form II polymorph of Lacosamide may exhibit two or more, three or more, four or more, five or more, six or more or seven characteristic X-ray powder diffraction peaks at about 5.2, 6.7, 12.6, 16.2, 20.0, 20.3, or 23.3 deg±0.2 degrees 2-theta.

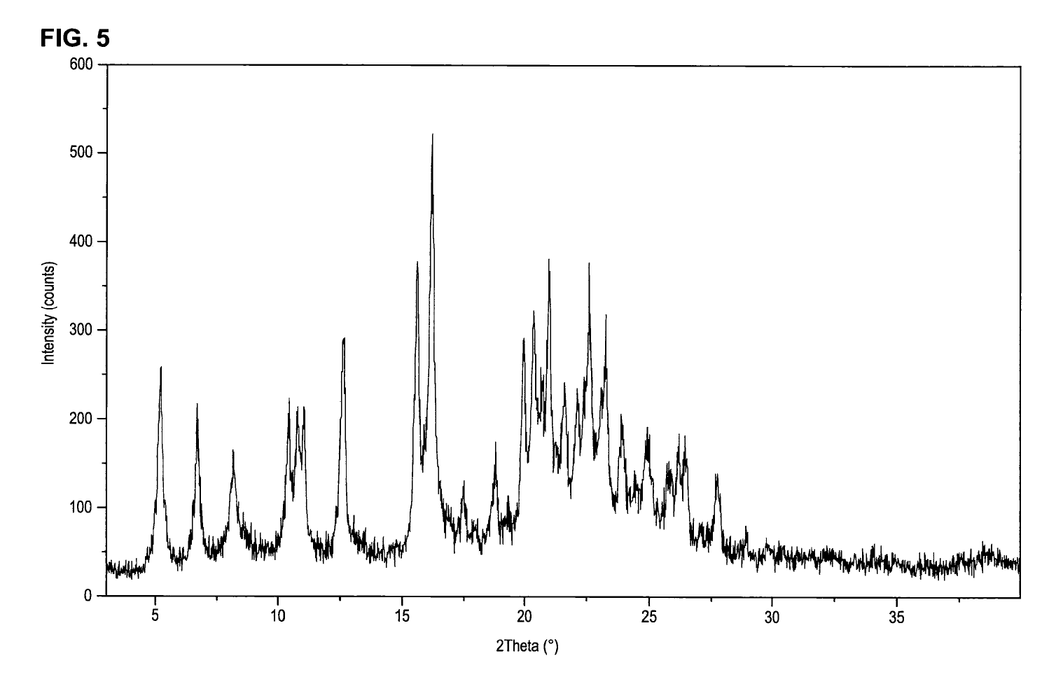

In a preferred embodiment, the present invention encompasses crystalline form II of Lacosamide characterized by data selected from a group consisting of a PXRD pattern having peaks at about 5.2, 6.7, 12.6, 16.2, 20.0 and 20.3±0.2 degrees 2-theta, a PXRD pattern as depicted in FIG. 5 and combination thereof.

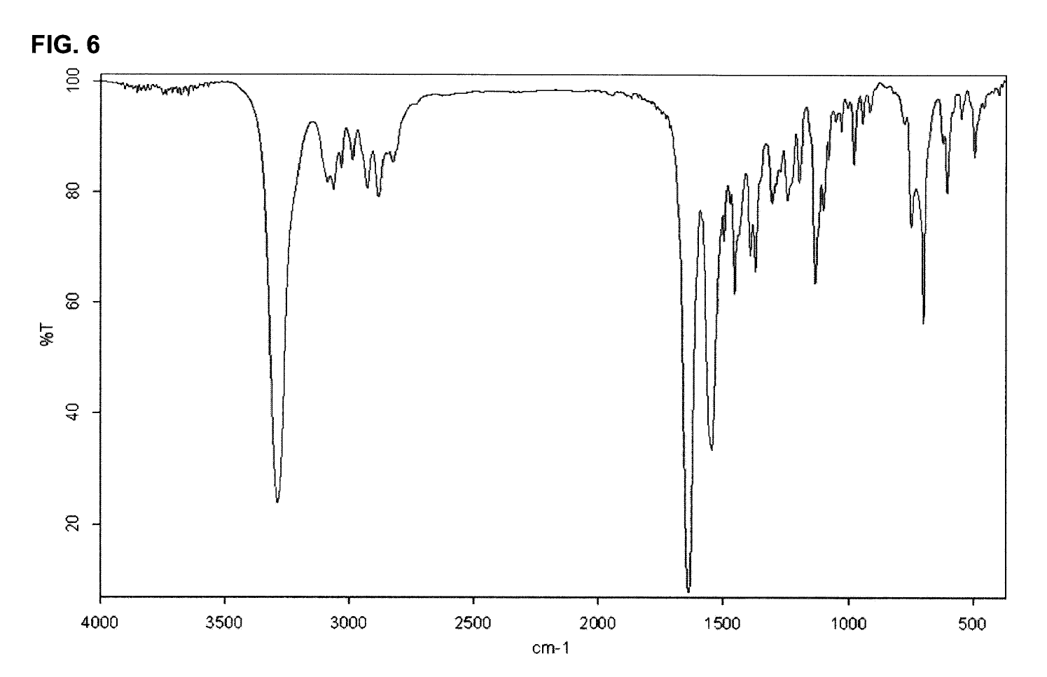

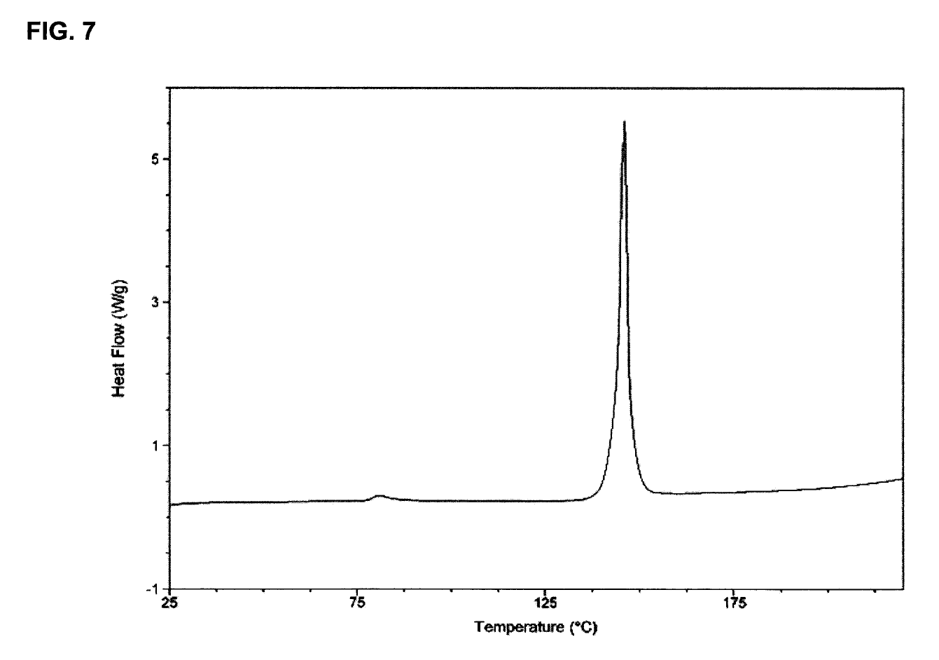

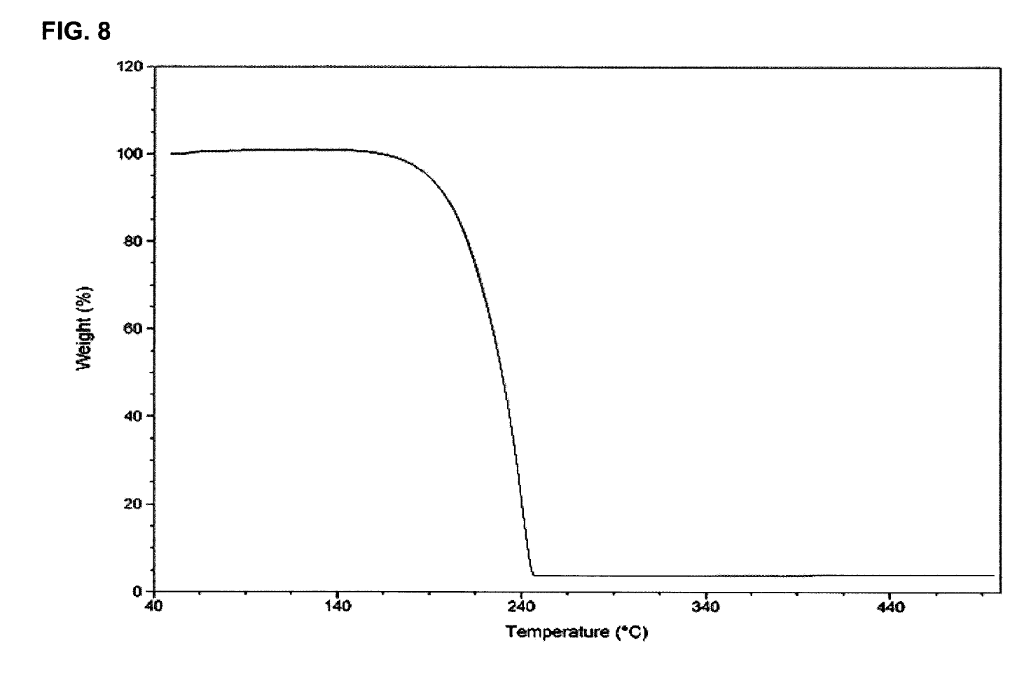

Crystalline form II can be further characterized by data selected from a group consisting of: a PXRD pattern having peaks at about 23.3deg±0.2 degrees 2-theta, an IR spectrum having absorbance peaks at about 3065, 2989, and 2883 cm⁻¹, an IR spectrum as depicted in FIG. 6, a DSC thermogram as shown in FIG. 7 and having two peaks at about 81° C. and about 146° C., and a thermal curve as measured by TGA as shown in FIG. 8.

In a preferred embodiment, the above form I is polymorphically pure.

As used herein the term polymorphically pure form II corresponds to composition containing Lacosamide form II and not more than about 10% by weight, preferably, not more than 5%, and more preferably, not more than 1% by weight, of Lacosamide form I.

The amount of Lacosamide form I and form II in the said composition can be measured by solid-state ¹³C NMR or PXRD. For example, the amount of form I can be measured by solid-state ¹³C NMR using the peak at 137.8 ppm±0.2; and the amount of form II can be measured by any one of the peaks at 5.2 and 16.2 degree±0.2 degrees 2-theta.

The above Lacosamide form II can be prepared by a process comprising providing a solution of Lacosamide in ethyl acetate and adding n-heptane to the obtained solution to obtain a suspension comprising the said crystalline form.

Typically, the said solution is obtained by combining Lacosamide and ethyl acetate and heating the said combination. Preferably, heating is done to a temperature of about 60° C. to about 70° C., more preferably, of about 70° C.

Preferably, the said solution is cooled to a temperature of about 50° C. to about 63° C., prior to the addition of n-heptane.

Further, the addition of n-heptane provides a suspension which is cooled to a temperature of about room temperature to about 0° C., prior to the recovery of the said crystalline form.

The above Lacosamide form II can be also prepared by a process comprising providing a solution of Lacosamide in ethyl acetate and cooling the solution to room temperature to obtain a suspension comprising the said crystalline form.

Preferably, the said solution is obtained as mentioned above, by combining Lacosamide and ethyl acetate and heating the said combination. Preferably, heating is done to a temperature of about 60° C. to about 70° C., more preferably, to about 70° C.

In a preferred embodiment, cooling is done rapidly, i.e. at a rate of about 1° C. per 1 minute. This means that the solution is not maintained at the above temperature, but cooled immediately.

The above processes for preparing crystalline lacosamide form II may further comprise recovery of the said crystalline form from the suspension. The recovery may be done, for example, by filtering the suspension comprising lacosamide form II, washing and drying.

Preferably, washing is done with ethyl acetate or a mixture of ethyl acetate and n-heptane. Preferably, drying is done at a temperature of about 50° C. Preferably, drying is done under vacuum.

The above form II of Lacosamide can be also prepared by a process comprising crystallizing Lacosamide from a solution comprising Lacosamide and a solvent selected from a group consisting of t-butanol, anisole, toluene or a mixture of two or more thereof.

In other embodiment, the solution consists of Lacosamide and a solvent selected from a group consisting of t-butanol, anisole, toluene or a mixture of two or more thereof.

Typically, the crystallization comprises providing a solution of Lacosamide in one or a mixture of two or more of the above solvent and cooling the said solution to obtain a suspension comprising the said crystalline form. The solution is preferably provided by combining Lacosamide with one or a mixture of two or more of the above solvent and heating the said combination. Preferably, heating is done to a temperature of about 50° C. to about 70° C., more preferably, to about 60° C. to about 70° C. Preferably, cooling is done to room temperature.

The above suspension may be further maintained for a period of about 2 hours, prior to the recovery of the said crystalline form.

The process for preparing crystalline lacosamide form II may further comprise recovery of the said crystalline form from the suspension. The recovery may be done, for example, by filtering the suspension comprising lacosamide form II and drying. Preferably, drying is done at room temperature for about overnight.

In another embodiment, the present invention provides crystalline Lacosamide form III (the form III polymorph). The form III polymorph of Lacosamide exhibits an X-ray powder diffraction pattern comprising one or more characteristic peaks at about 16.9, 23.0 and 31.0 deg±0.2 degrees 2-theta. For example, the form III polymorph of Lacosamide may exhibit two or three peaks at 16.9, 23.0 and 31.0 deg±0.2 degrees 2-theta.

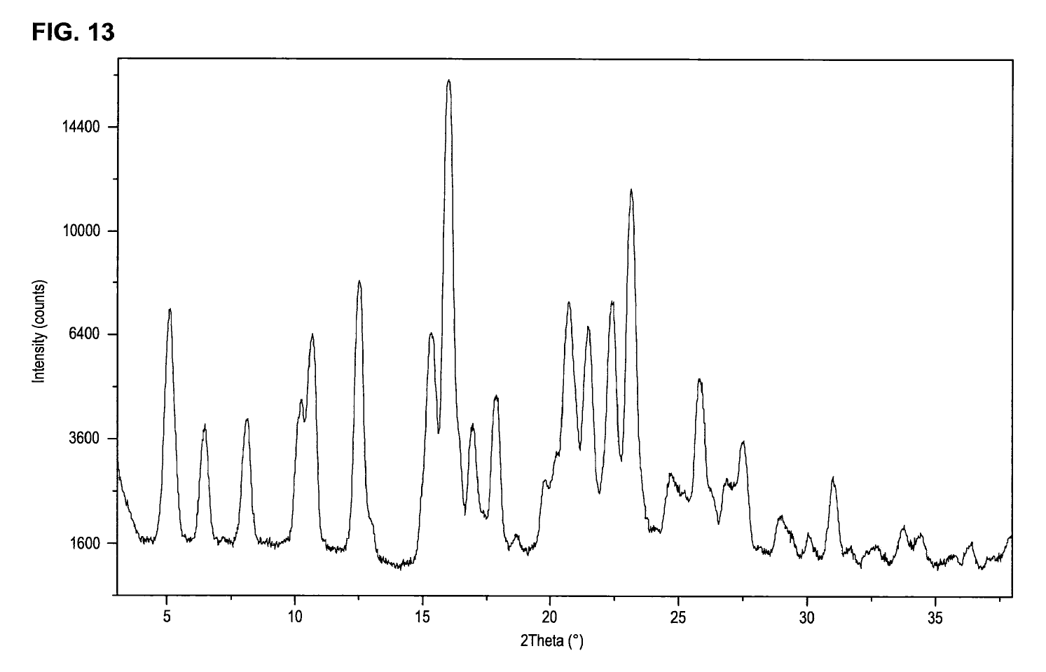

In a preferred embodiment, the present invention encompasses crystalline form III of Lacosamide characterized by data selected from a group consisting of a PXRD pattern having peaks at about 16.9, 23.0 and 31.0 deg±0.2 degrees 2-theta, a PXRD pattern as depicted in FIG. 13 and combination thereof.

The above Lacosamide form III can be prepared by a process comprising heating crystalline form II of Lacosamide to a temperature of at least about 85° C.

In another embodiment, the present invention encompasses amorphous Lacosamide.

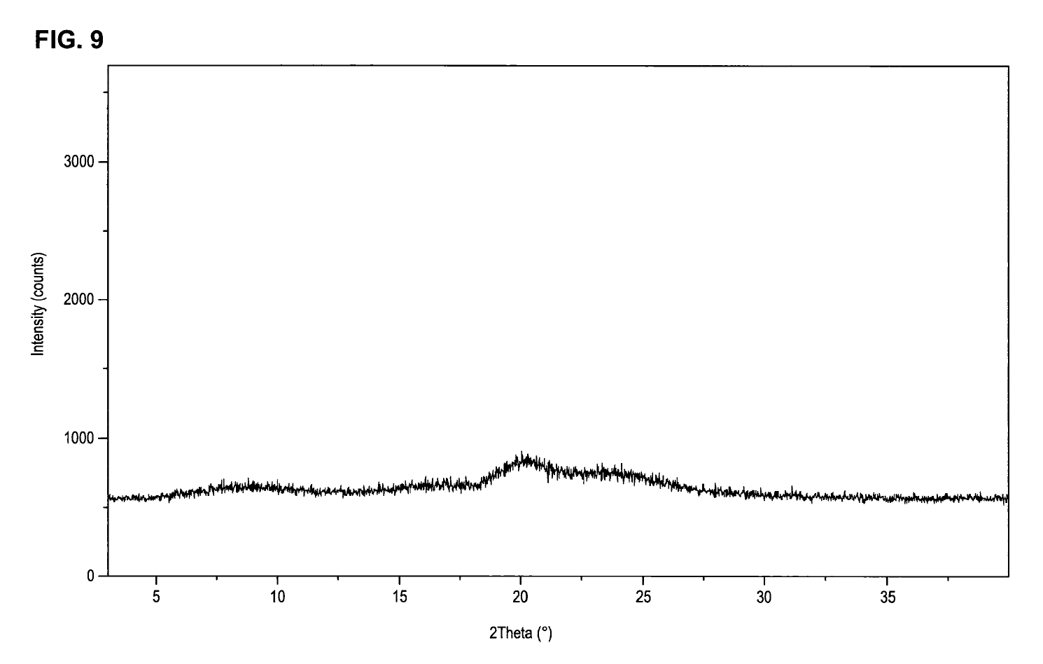

The above amorphous Lacosamide can be characterized by a PXRD pattern as depicted in FIG. 9, wherein it is characterized by lack of crystalline peaks in the XRD pattern.

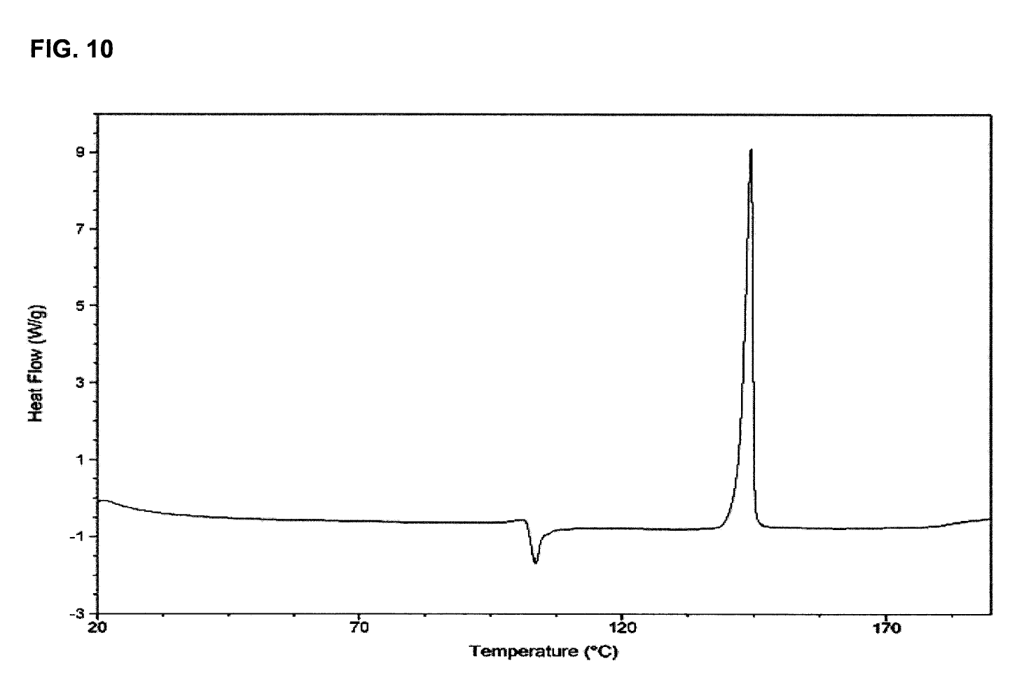

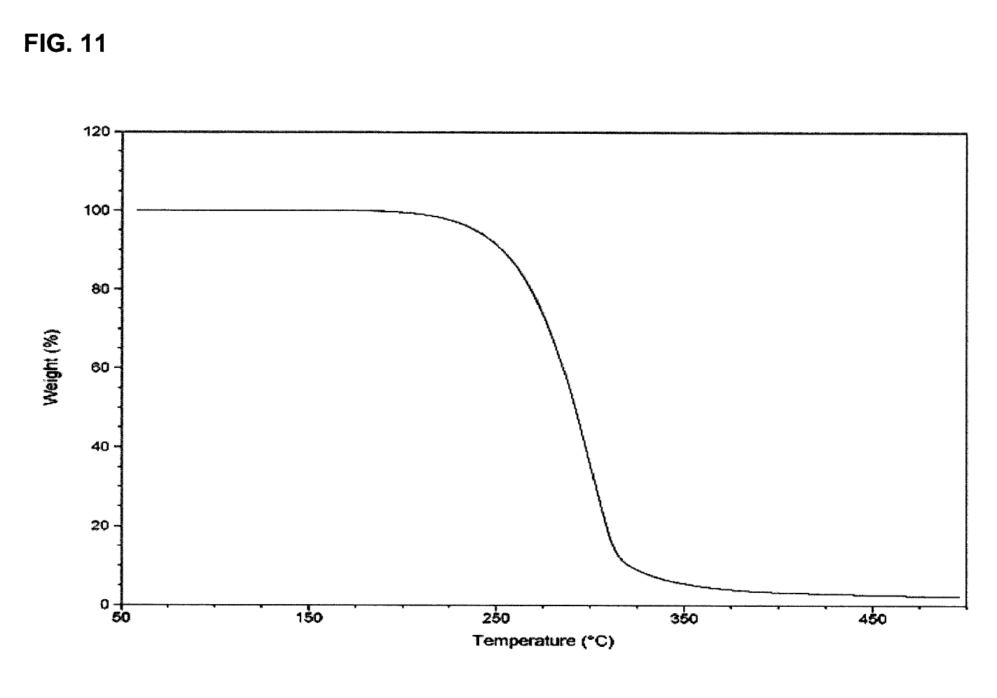

Amorphous Lacosamide can be farther characterized by data selected from a group consisting of: a DSC thermogram as shown in FIG. 10 and having two peaks at about 104° C. and at about 144° C. and a thermal curve as measured by TGA, as shown in FIG. 11.

Amorphous Lacosamide can be prepared in several different ways. For example an aqueous solution of Lacosamide may be lyophilized; i.e., the aqueous solution of Lacosamide is frozen and placed under vacuum on a freeze dryer until all of the water and any co-solvent is removed by sublimation. Typically the aqueous solution comprises 100% water. However, the skilled artisan will understand that small amounts of organic co-solvents may be used in the aqueous solution so long as they do not interfere with the lyophilization.

Amorphous Lacosamide may also be prepared by dissolving Lacosamide in t-butyl alcohol and removing the solvent under reduced pressure by, e.g., rotary evaporation. Amorphous Lacosamide may also be melted on a glass plate and quickly cooled, e.g., by ice quench.

The above forms of Lacosamide can be used to prepare pharmaceutical compositions.

In one embodiment, the present invention provides pharmaceutical compositions comprising at least one of the above forms of Lacosamide and pharmaceutically acceptable excipient.

In another embodiment, the invention encompasses a pharmaceutical composition comprising at least one of the above described polymorphic and amorphous forms of Lacosamide prepared according to the processes of the present invention, and at least one pharmaceutically acceptable excipient.

In another embodiment, the invention encompasses a process for preparing a pharmaceutical composition comprising at least one of the above-described polymorphic and amorphous forms of Lacosamide, and at least one pharmaceutically acceptable excipient.

In another embodiment, the invention encompasses the use of at least one of the above described polymorphic and amorphous form of Lacosamide for the manufacture of a medicament for the treatment of e.g., as an anticonvulsant or for relieving pain

The pharmaceutical composition of the present invention can be in a solid or a non-solid form. If the pharmaceutical composition is in a non-solid form, the polymorphic and amorphous form of Lacosamide, within the composition, are retained as solid(s) in the non-solid pharmaceutical composition, e.g., as a suspension, foam, ointment and etc.

The pharmaceutical composition can be used to make appropriate dosage forms such as tablets, powders, capsules, suppositories, sachets, troches and losenges.

The polymorphic and amorphous form of Lacosamide, of the present invention, particularly in a pharmaceutical composition and dosage form, can be used to treat relieving pain, in a mammal such as a human, comprising administering a treatment effective amount of the polymorphic and amorphous form of Lacosamide in the mammal. The treatment effective amount or proper dosage to be used can be determined by one of ordinary skill in the art, which can depend on the method of administration, the bioavailability, the age, sex, symptoms and health condition of the patient, and the severity of the disease to be treated, etc.

In yet another embodiment, the present invention encompasses a method of treating central nervous system disorders and alleviating pain comprising administering to a subject in need thereof of a pharmaceutical composition comprising at least one of the above-described polymorphic and amorphous forms of Lacosamide, and at least one pharmaceutically acceptable excipient. In one embodiment, the method include administering to a subject suffering from a central nervous system disorder an anti-convulsant effective amount of any of the forms of Lacosamide disclosed herein. For example, the disorder may be epilepsy.

In another embodiment the methods include administering to a subject suffering from neuropathic pain a pain-reducing effective amount of any of the forms of Lacosamide disclosed herein. For example, the neuropathic pain can be diabetic neuropathic pain. In other embodiments, the methods include administering to a subject suffering from migraine headache a headache-reducing effective amount of any of the forms of Lacosamide disclosed herein.

In another embodiment, the present invention encompasses an amorphous composition containing Lacosamide combined with a pharmaceutical acceptable ingredient, wherein the pharmaceutical acceptable ingredient is selected from a group consisting of Hypromellose or Hydroxypropyl cellulose.

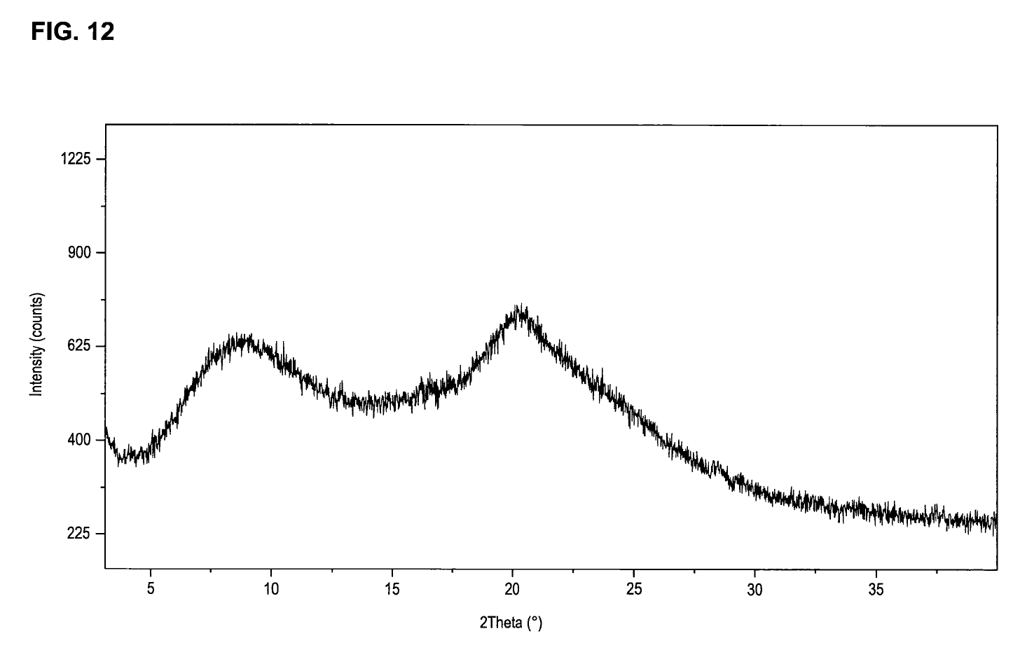

The amorphous composition containing Lacosamide combined with Hypromellose can be characterized by PXRD pattern as depicted in FIG. 12.

The above amorphous composition can be prepared by a process comprising spray drying a solution comprising Lacosamide and a pharmaceutical acceptable ingredient and water, wherein the pharmaceutical acceptable ingredient is selected from a group consisting of Hypromellose or Hydroxypropyl cellulose.

Preferably, the solution consists of Lacosamide, water and a pharmaceutical acceptable ingredient.

Preferably, the said solution is obtained by combining the pharmaceutical acceptable ingredient, Lacosamide and water at room temperature.

Preferably, spray drying is done at a temperature of about 110° C. to about 120° C.

The present invention, thus generally described, will be understood more readily by reference to the following examples, which are provided by way of illustration and are not intended to be limiting of the present invention.

### EXAMPLES

The crude Lacosamide used as starting material in the following examples may be prepared by known methods such as, e.g., those described in U.S. Pat. No. 6,048,899 to Kohn and Andurkar and U.S. Patent Application Publication No. 2008/0027137 to Riedner and Dunne.

### X-Ray Powder Diffraction

Samples after being powdered in a mortar and pestle are applied directly on silicon plate holder. The X-ray powder diffraction pattern was measured with Philips X'Pert PRO X-ray powder diffractometer, equipped with Cu irradiation source=1.54060 [symbol] ( [symbol] ngstrom), X'C.elerator (2.022° 2Θ) detector. Scanning parameters: angle range: 3-40 deg., step size 0.0167, time per step 100 s, continuous scan. The accuracy of peak positions was defined as ±0.2 degrees due to experimental differences like instrumentations and sample preparations.

Temperature dependent X-ray powder diffraction (TDXD) was carried out with a Bruker AXS D8 θ-θ powder diffractometer with parafocusing Bragg-Brentano geometry using CuK_{α} radiation (λ=1.5418 Å, U=34 kV, I=20 mA). The sample was placed on a Pt/Rh ribbon in a MRI high-temperature oven-camera and VPSlits were used to fix the constant irradiated length (20 mm). Data were scanned with an ultrafast detector LynxEye over the angular range 3-38° (2θ) with a step size of 0.0196° (2θ) and a counting time of 58.6 s step⁻¹. The heating and cooling process was controlled by Eurotherm 2404.

### Infrared Spectra

IR spectra of Lacosamide forms I and II were obtained by using a KBr pellet and Spectrum GX manufactured by Perkin-Elmer and are shown in FIGS. 2, and 6, respectively. The standard error for absorption band maximums is ±4 cm⁻¹.

### DSC Method

DSC analysis was performed on Q 1000 MDSC TA instruments with heating rate of 10° C./min, under nitrogen flow of 50 ml/min. Standard aluminum, closed pan was used, sample mass was about 1-5 mg.

### TGA Method

TG analysis was performed under flow of nitrogen (60 ml/min) on TGA 2950 TA instrument, with heating rate of 10° C./min. Open platinum pan was used , sample mass was about 10 mg.

### Solid State NMR

Instruments Parameters:
¹³CNMR at 125 MHz using Bruker Avance II+ 500
SB probe using 4 mm rotors
Magic angle was set using KBr
Homogeneity of magnetic field checked using adamantane
Parameters for Cross polarization optimized using glycine
Spectral reference set according to glycine as external standard (176.03 ppm for low field carboxyl signal)

### Scanning Parameters Used:

Magic Angle Spinning Rate: 11 kHz
Pulse Program: cp with tppm 15 during decoupling
Delay time: 2 seconds
Number of Scans: 2048
Standard error for solid state NMR peaks is ±0.2 ppm.

### Example 1

### Form I Lacosamide

Crude Lacosamide (14.3 g) was dissolved in a mixture of 10 mL of CH₂Cl₂ and 120 mL of ethyl acetate while heating to reflux. Upon cooling to room temperature, the product crystallized from solution. The resulting suspension of crystals was stirred for 3 hours at room temperature, and additional 2 hours at 5° C. The suspension was filtered to provide 12.3 g of white crystalline product, identified as form I Lacosamide.

### Example 2

### Form I Lacosamide

Crude Lacosamide (100 mg) was dissolved in 820 μL of ethyl acetate while heating to reflux. Upon cooling to room temperature, the product crystallized from solution. The resulting suspension of crystals was stirred for 3 hours at room temperature and an additional 2 hours at 5° C. The suspension was filtered to provide 77 mg of white crystalline product, identified as form I Lacosamide.

### Example 3

### Form I Lacosamide

Crude Lacosamide (20 mg) was dissolved in 1 mL of acetone while heating. The resulting solution was placed in closed bottle at room temperature to crystallize over 5-7 days. A suspension of crystals was obtained and was filtered and dried over night at room temperature and pressure. The resulting white crystalline solid (13 mg) was identified as form I Lacosamide.

### Example 4

### Form I Lacosamide

2.1 g of Lacosamide was dissolved in 30 ml of ethyl acetate by heating. Prepared solution was dropwisely added to 30 ml of n-heptane at room temperature. Crystallization started immediately. After addition was completed, suspension was stirred for 0.5 hour and crystals were filtered, washed with ethyl acetate/heptane 1:1 and dried at 50° C./vacuo yielding 1.8 g of Lacosamide form I.

### Example 5

### Form I Lacosamide

2.1 g of Lacosamide was dissolved in 30 ml of ethyl acetate by heating. Prepared solution was dropwisely added to 30 ml of n-heptane at 50° C. Crystallization started immediately. After addition was completed, suspension was cooled down to room temperature. Crystals were filtered, washed with ethyl acetate/heptane 1:1 and dried at 50° C./vacuo yielding 1.8 g of Lacosamide form I.

### Example 6

### Form I Lacosamide

2.1 g of Lacosamide was dissolved in 30 ml of ethyl acetate by heating. Prepared solution was dropwisely added to 30 ml of n-heptane previously seeded with crystals of form I at room temperature. Crystallization started immediately. After addition was completed, suspension was stirred for 0.5 hour and crystals were filtered and dried at 50° C./vacuo, yielding 1.8 g of Lacosamide form I.

### Example 7

### Form I Lacosamide

2.1 g of Lacosamide was dissolved in 30 ml of ethyl acetate by heating. Prepared solution was dropwisely added to 30 ml of n-heptane previously seeded with crystals of form II at room temperature. Crystallization started immediately. After addition was completed, suspension was stirred for 0.5 hour and crystals were filtered and dried at 50° C./vacuo, yielding 1.9 g of Lacosamide form I.

### Example 8

### Form I Lacosamide

13.5 g of Lacosamide was dissolved in 150 ml of ethyl acetate by heating. Solution crystallized by cooling at about 52° C. Suspension was stirred at 40° C. for about 4 hours, cooled to 20° C. and stirred for another 0.5 hour. Crystals were filtered, washed with ethyl acetate and dried at 50° C./vacuo yielding 10.9 g Lacosamide form I.

### Example 9

### Form I Lacosamide

13.5 g of Lacosamide was dissolved in 150 ml of ethyl acetate by heating. Solution crystallized by cooling at about 53° C. Suspension was stirred at 50° C. for about 4 hours, cooled to 20° C. and stirred for another 0.5 hour. Crystals were filtered, washed with ethyl acetate and dried at 50° C./vacuo yielding 10.9 g Lacosamide form I.

### Example 10

### Form I Lacosamide

2.0 g of Lacosamide was dissolved in 22 ml of ethyl acetate by heating. Solution was filtered to flask pre-heated at 70° C. and cooled to 40° C. Obtained suspension was diluted with 5 ml of ethyl acetate, stirred at 40° C. for 4 hours and cooled to 20° C. Crystals were filtered, washed with ethyl acetate and dried at 50° C./vacuo yielding 1.5 g Lacosamide form I.

### Example 11

### Form I Lacosamide

26.9 g of Lacosamide was dissolved in 310 ml of ethyl acetate by heating. Solution crystallized by cooling. Suspension was stirred at 40° C. for about 4 hours, cooled to 20° C. and stirred for another 3 hours. Crystals were filtered, washed with ethyl acetate/heptane 1:1 and dried at 50° C./vacuo yielding 20.5 g Lacosamide form I.

### Example 12

### Form I Lacosamide

27.0 g of Lacosamide was dissolved in 300 ml of ethyl acetate by heating. Solution crystallized by cooling at about 46° C. Suspension was stirred at 40° C. for about 4 hours, cooled to 4° C. and stirred for another 0.5 hour. Crystals were filtered, washed with cold ethyl acetate and dried at 50° C./vacuo yielding 23.0 g Lacosamide form I.

### Example 13

### Form I Lacosamide

30.0 g of Lacosamide was dissolved in 300 ml of ethyl acetate by heating. Solution crystallized by cooling at about 47° C. Suspension was stirred at 40° C. for about 4 hours, cooled to 4° C. and stirred for another 0.5 hour. Crystals were filtered, washed with cold ethyl acetate and dried at 50° C./vacuo yielding 24.4 g Lacosamide form I.

### Example 14

### Form I Lacosamide

10.0 g of Lacosamide was dissolved in 78 ml of ethyl acetate and 2.24 ml of water by heating. Solution crystallized by cooling at about 37° C. Suspension was stirred at 30° C. for about 4 hours, cooled to room temperature and stirred for another 0.5 hour. Crystals were filtered and dried at 50° C./vacuo yielding 6.0 g Lacosamide form I.

### Example 15

### Form I Lacosamide

10.0 g of Lacosamide was dissolved in 64.5 ml of ethyl acetate and 1.85 ml of water by heating. Solution crystallized by cooling at about 24° C. Suspension was stirred at room temperature for about 4 hours. Crystals were filtered and dried at 50° C./vacuo yielding 6.3 g Lacosamide form I.

### Example 16

### Form I Lacosamide

5.0 g of Lacosamide was dissolved in 39.5 ml of ethyl acetate and 0.5 ml of water by heating. Solution crystallized by cooling at about 48° C. Suspension was stirred at 40° C. for about 4 hours, cooled to room temperature and stirred for another 0.5 hour. Crystals were filtered, washed with cold ethyl acetate and dried at 50° C./vacuo yielding 3.6 g Lacosamide form I.

### Example 17

### Form I Lacosamide

5.0 g of Lacosamide was dissolved in 32.8 ml of ethyl acetate and 0.5 ml of water by heating. Solution crystallized by cooling at about 48° C. Suspension was stirred at 40° C. for about 4 hours, cooled to room temperature and stirred for another 0.5 hour. Crystals were filtered, washed with cold ethyl acetate and dried at 50° C./vacuo yielding 3.7 g Lacosamide form I.

### Example 18

### Form I Lacosamide

5.0 g of Lacosamide was dissolved in 39.6 ml of ethyl acetate and 0.4 ml of water by heating. Solution crystallized by cooling at about 52° C. Suspension was stirred at 40° C. for about 4 hours, cooled to room temperature and stirred for another 0.5 hour. Crystals were filtered, washed with cold ethyl acetate and dried at 50° C./vacuo yielding 3.8 g Lacosamide form I.

### Example 19

### Form I Lacosamide

5.0 g of Lacosamide was dissolved in 39.7 ml of ethyl acetate and 0.3 ml of water by heating. Solution crystallized by cooling. Suspension was stirred at 40° C. for about 4 hours, cooled to room temperature and stirred for another 0.5 hour. Crystals were filtered, washed with cold ethyl acetate and dried at 50° C./vacuo yielding 3.7 g Lacosamide form I.

### Example 20

### Form I Lacosamide

5.0 g of Lacosamide was dissolved in 39.8 ml of ethyl acetate and 0.2 ml of water by heating. Solution crystallized by cooling at about 50° C. Suspension was stirred at 40° C. for about 4 hours, cooled to room temperature and stirred for another 0.5 hour. Crystals were filtered, washed with cold ethyl acetate and dried at 50° C./vacuo yielding 3.8 g Lacosamide form I.

### Example 21

### Form I Lacosamide

2.0 g of Lacosamide was dissolved in 16 ml of ethyl acetate and 0.1 ml of water by heating. Solution was filtered in flask pre-heated at 70° C. Solution crystallized by cooling. Suspension was stirred at 40° C. for about 4 hours, cooled to room temperature and stirred for another hour. Crystals were filtered, washed with cold ethyl acetate and dried at 50° C./vacuo yielding 1.5 g Lacosamide form I.

### Example 22

### Form II Lacosamide

Crude Lacosamide (20 mg) was dissolved in 1 mL of selected tert-butanol while heating. The resulting solution was placed in a closed bottle at room temperature to crystallize. The resulting product was filtered and dried over night at room temperature. The product was identified as form II Lacosamide.

### Example 23

### Form II Lacosamide

Crude Lacosamide (100 mg) was dissolved in 2 mL of anisole while heating. Product crystallizes while cooling to room temperature. Obtained suspension was stirred for another 2 hours. By filtration 68 mg of product identified as form II Lacosamide was obtained.

### Example 24

### Form II Lacosamide

Crude Lacosamide (1 g) was dissolved in 15 mL of toluene while heating. Product crystallizes while cooling to room temperature. Obtained suspension was stirred for another 2 hours. By filtration 0.74 g of product, identified as form II Lacosamide was obtained.

### Example 25

### Form II Lacosamide

3 g of Lacosamide was dissolved in 60 ml of ethyl acetate by heating. Solution was cooled and crystallization occurred at about 18° C. Crystals were filtered and dried at 50° C./vacuo yielding 2.1 g of Lacosamide form II.

### Example 26

### Form II Lacosamide

4.2 g of Lacosamide was dissolved in 60 ml of ethyl acetate by heating. Solution crystallized by cooling to room temperature. Crystals were filtered, washed with ethyl acetate and dried at 50° C./vacuo yielding 3.3 g of Lacosamide form II.

### Example 27

### Form II Lacosamide

4.8 g of Lacosamide was dissolved in 60 ml of ethyl acetate by heating. Solution was cooled by 1° C./min to room temperature. Crystals were filtered, washed with ethyl acetate and dried at 50° C./vacuo yielding 4.0 g of Lacosamide form II.

### Example 28

### Form II Lacosamide

18.0 g of Lacosamide was dissolved in 200 ml of ethyl acetate by heating in 0.5 L flask. Solution was filtered into 0.5 L reactor pre-heated at 70° C. Solution was cooled to about 47° C. and seeded with 50 mg of lacosamide (mixture of forms I and II). Crystallization started 10 minutes later. Suspension was stirred for another 15 minutes at 47° C. and cooled down to room temperature. Crystals were filtered and dried at 50° C./vacuo yielding 13.9 g of Lacosamide form II.

### Example 29

### Form II Lacosamide

5.4 g of Lacosamide was dissolved in 60 ml of ethyl acetate by heating. Solution was cooled by 1° C./min to room temperature. Crystals were filtered, washed with ethyl acetate and dried at 50° C./vacuo yielding 4.5 g of Lacosamide form II.

### Example 30

### Form II Lacosamide

2.7 g of Lacosamide was dissolved in 30 ml of ethyl acetate by heating. Solution crystallized by cooling at about 53° C. Suspension was stirred at 53° C. for about 0.5 hour and cooled to room temperature. Crystals were filtered and dried at 50° C./vacuo yielding 2.1 g of Lacosamide form II.

### Example 31

### Form II Lacosamide

6.0 g of Lacosamide was dissolved in 60 ml of ethyl acetate by heating. Solution was cooled by 1° C./min to room temperature. Crystals were filtered, washed with ethyl acetate and dried at 50° C./vacuo yielding 5.2 g of Lacosamide form II.

### Example 32

### Form II Lacosamide

54.8 g of Lacosamide was dissolved in 700 ml of ethyl acetate. Solution crystallized by cooling at about 45° C. Suspension was stirred at 40° C. for about 1.5 hours, cooled to 20° C. and stirred for another 0.5 hour. Crystals were filtered, washed with ethyl acetate and dried at 50° C./vacuo yielding 42.6 g Lacosamide form II.

### Example 33

### Form II Lacosamide

1.0 g of Lacosamide was dissolved in 27 ml of ethyl acetate by heating. Solution was cooled to 50° C. and 27 ml of n-heptane was dropwisely added. Few minutes after crystallization started. Suspension was cooled to 0-5° C. and stirred for 15 minutes. Crystals were filtered, washed with ethyl acetate/heptane 1:1 and dried at 50° C./vacuo yielding 0.95 g of Lacosamide form II.

### Example 34

### Form II Lacosamide

3.0 g of Lacosamide was dissolved in 40 ml of ethyl acetate by heating. Solution was cooled to 55° C. and 20 ml of n-heptane was dropwisely added. Solution crystallized by further cooling at 49° C. Suspension was cooled to room temperature. Crystals were filtered, washed with ethyl acetate/heptane 1:1 and dried at 50° C./vacuo yielding 2.8 g of Lacosamide form II.

### Example 35

### Form II Lacosamide

15.0 g of Lacosamide was dissolved in 200 ml of ethyl acetate by heating to reflux in 0.5 L flask. Solution was filtered to 0.5 L glass reactor pre-heated at 70° C. 50 ml of n-heptane was dropwisely added at this temperature. Solution crystallized by cooling at 60° C. Suspension was cooled to room temperature. Crystals were filtered and dried at 50° C./vacuo yielding 12.9 g of Lacosamide form II.

### Example 36

### Form II Lacosamide

3.0 g of Lacosamide was dissolved in 50 ml of ethyl acetate by heating. Solution was cooled to 55° C. and 10 ml of n-heptane was dropwisely added. Solution crystallized by further cooling at 46° C. Suspension was cooled to room temperature. Crystals were filtered, washed with ethyl acetate/heptane 1:1 and dried at 50° C./vacuo yielding 2.5 g of Lacosamide form II.

### Example 37

### Form II Lacosamide

19.4 g of Lacosamide was dissolved in 215 ml of ethyl acetate by heating. Solution was cooled to 55° C. and 85 ml of n-heptane was dropwisely added within 10 minutes. Solution crystallized after few minutes at 50° C. Suspension was cooled to 40° C., stirred at this temperature for 4 hours, cooled to room temperature and stirred for another 3 hours. Crystals were filtered, washed with cold ethyl acetate/heptane 1:1 and dried at 50° C./vacuo yielding 14.8 g of Lacosamide form II.

### Example 38

### Form II Lacosamide

Solution of 2.1 g of Lacosamide in 30 ml of ethyl acetate was dropwisely added to mixture of 10 ml ethyl acetate and 20 ml n-heptane at room temperature. So obtained crystals were filtered and dried at 50° C./vacuo yielding 1.7 g of Lacosamide form II.

### Example 39

### Form II Lacosamide

2.7 g of Lacosamide was dissolved in 52 ml of methyl isobutyl ketone by heating. Solution was filtered and cooled. Crystallization occurred at 63° C. Suspension was stirred at 63° C. for about 0.5 hour and cooled to room temperature. Crystals were filtered and dried at 50° C./vacuo yielding 2.3 g of Lacosamide form II.

### Example 40

### Form II Lacosamide

2.7 g of Lacosamide was dissolved in 52 ml of methyl isobutyl ketone by heating. Solution was filtered and 10 ml of n-heptane was added dropwisely. Crystallization occurred at 55° C. Suspension was stirred at 55° C. for about 0.5 hour and cooled to room temperature. Crystals were filtered and dried at 50° C./vacuo yielding 2.3 g of Lacosamide form II.

### Example 41

### Amorphous Lacosamide

Crude Lacosamide (30 mg) was dissolved at room temperature in 1.5 mL of H₂O and freeze-dried. 29 mg of white product was obtained and identified as amorphous Lacosamide.

### Example 42

### Amorphous Lacosamide

Crude Lacosamide (30 mg) was melted on glass plate using a Koffler bench, at 147° C. The melt was quench cooled on ice. 30 mg of amorphous Lacosamide was obtained.

### Example 43

### Amorphous Lacosamide

Crude Lacosamide (50 mg) was dissolved in 3 mL of tert-butanol at room temperature. The solvent was removed under reduced pressure and 22 mg of amorphous Lacosamide was obtained.

### Example 44

### Mixture of Form I and II of Lacosamide

2.7 g of Lacosamide was dissolved in 36 ml of ethyl acetate and 9 ml of n-heptane by heating. Solution crystallized by cooling at about 49° C. Suspension was cooled to room temperature and stirred for 0.5 hour. Crystals were filtered and dried at 50° C./vacuo yielding 2.2 g Lacosamide mixture of form I and form II.

### Example 45

### Mixture of Form I and II of Lacosamide

3.0 g of Lacosamide was dissolved in 30 ml of ethyl acetate by heating. Solution was cooled to 55° C. and 30 ml of n-heptane was dropwisely added. Solution crystallized before all heptane was added. Suspension was cooled to room temperature, crystals were filtered, washed with cold ethyl acetate/heptane 1:1 and dried at 50° C./vacuo yielding 2.9 g of Lacosamide mixture of form I and form II.

### Example 46

### Mixture of Form I and II of Lacosamide

20.0 g of Lacosamide was dissolved in 250 ml of ethyl acetate by heating. Solution was filtered to flask pre-heated at 70° C. and cooled down to 40° C. Solution crystallized at about 47° C. Suspension was stirred at 40° C. for about 3 hours and cooled to room temperature. Crystals were filtered, washed with cold ethyl acetate and dried at 50° C./vacuo yielding 15.4 g Lacosamide mixture of form I and form II.

### Example 47

### Mixture of Form I and II of Lacosamide

5.0 g of Lacosamide was dissolved in 65 ml of n-butyl acetate by heating. Solution crystallized by cooling at about 60° C. Suspension was stirred at 40° C. for about 3 hours and cooled to room temperature. Crystals were filtered, washed with cold n-butyl acetate and dried at 50° C./vacuo yielding 4.2 g Lacosamide mixture of form I and form II.

### Example 48

### Mixture of Form I and II of Lacosamide

5.0 g of Lacosamide was dissolved in 65 ml of i-butyl acetate by heating. Solution crystallized by cooling at about 63° C. Suspension was stirred at 40° C. for about 3 hours and cooled to room temperature. Crystals were filtered, washed with cold i-butyl acetate and dried at 50° C./vacuo yielding 4.3 g Lacosamide mixture of form I and form II.

### Example 49

### Interconversion of Form II to Form I

27.0 g of Lacosamide was dissolved in 300 ml of ethyl acetate. Solution crystallized by cooling at about 42° C. Suspension was cooled to 40° C. and samples were taken in time intervals, filtered and crystal form was determined using XRPD. Sample taken immediately when temperature reached 40° C. showed to be form II. Sample taken after 2 hours at 40° C. showed to be mixture of form I and form II. Sample taken after 4 hours at 40° C. showed to be form I. After 5 hours of stirring at 40° C., suspension was cooled to 20° C. and stirred for about 18 hours. Crystals were filtered, washed with ethyl acetate and dried at 50° C./vacuo yielding 20.0 g Lacosamide form I.

### Example 50

### Inter Conversion of Form II to Form I

30.0 g of Lacosamide was dissolved in 239 ml of ethyl acetate and 1 ml of water. Solution crystallized by cooling at about 46° C. Suspension was cooled to 40° C. and samples were taken in time intervals, filtered and crystal form was determined using XRPD. Sample taken immediately when temperature reached 40° C. showed to be mixture of form I and form II. Sample taken after 2 hours at 40° C. showed to be form I. After 4 hours of stirring at 40° C., suspension was cooled to 20° C. Crystals were filtered, washed with cold ethyl acetate and dried at 50° C./vacuo yielding 22.9 g Lacosamide form I.

### Example 51

### Amorphous Composition Containing Lacosamide Combined with Hypromellose

Hypromellose (100 mg) was dissolved in water (40 ml) at room temperature. Lacosamide (100 mg) was added to the solution and dissolved, at room temperature. Obtained solution was filtrated and spray dried at 110-120° C. Isolated white crude product (100 mg) was analyzed by XRPD and found to be amorphous form.

### Example 52

### Amorphous Composition Containing Lacosamide Combined with Hydroxypropyl Cellulose

Hydroxypropyl cellulose (100 mg) was dissolved in water (40 ml) at room temperature. Lacosamide (100 mg) was added to the solution and dissolved, at room temperature. Obtained solution was filtrated and spray dried at 110-120° C. Isolated white crude product (100 mg) was analyzed by XRPD and found to be amorphous form.

### Example 53

### Form III of Lacosamide

The sample of form II lacosamide (50 mg) was placed on a Pt/Rh ribbon in a MRI high-temperature oven-camera, and heated at rate of 10° C./min up to 100° C. The heating and cooling process was controlled by Eurotherm 2404. Transformation of form II to form III occurs around 80° C., and is completely reversible as the sample is cooled back to room temperature.

1. A crystalline form of Lacosamide characterized by data selected from the group consisting of a PXRD pattern having peaks at about 8.3, 13.0, 16.6, 17.7 and 21.4 deg±0.2 degrees 2-theta, a PXRD pattern as depicted in FIG. 1, and combinations thereof.

2. The crystalline form of Lacosamide of claim 1, characterized by a PXRD pattern having peaks at about 8.3, 13.0, 16.6, 17.7 and 21.4 deg±0.2 degrees 2-theta.

3. The crystalline form of Lacosamide of claim 1, characterized by a PXRD pattern as depicted in FIG. 1.

4. The crystalline form of Lacosamide of claim 1, further characterized by a PXRD pattern having peaks at about 24.9 and 25.4 deg±0.2 degrees 2-theta.

5. A composition containing the crystalline form of Lacosamide of claim 1 and not more than about 10% by weight of a crystalline form of Lacosamide characterized by a PXRD pattern having peaks at about 5.2, 6.7, 12.6, 16.2, 20.0 and 20.3±0.2 degrees 2-theta.

6. A process for preparing a crystalline form of Lacosamide characterized by data selected from a group consisting of a PXRD pattern having peaks at about 8.3, 13.0, 16.6, 17.7 and 21.4 deg±0.2 degrees 2-theta, a PXRD pattern as depicted in FIG. 1, and combinations thereof, comprising:
providing a solution of Lacosamide in ethyl acetate; and
adding the obtained solution to n-heptane to obtain a suspension comprising the said crystalline form.

7. The process of claim 6, wherein the solution is prepared by combining Lacosamide and ethyl acetate and heating the combination.

8. The process of claim 7, wherein the heating is done to a temperature of about 60° C. to about 70° C.

9. A process for preparing a crystalline form of Lacosamide characterized by data selected from a group consisting of a PXRD pattern having peaks at about 8.3, 13.0, 16.6, 17.7 and 21.4 deg±0.2 degrees 2-theta, a PXRD pattern as depicted in FIG. 1 and combinations thereof, comprising:
providing a suspension of Lacosamide in ethyl acetate and maintaining the obtained suspension for at least 4 hours at a temperature of about 40° C. to about 50° C.

10. The process of claim 9, wherein the said suspension is obtained, by combining Lacosamide and ethyl acetate, heating the said combination to a temperature of about 60° C. to about 70° C. to obtain a solution and cooling the obtained solution to a temperature of about 40° C. to about 50° C.

11. The process of claim 10, wherein the heating is done to a temperature of about 70° C.

12. The process of claim 9, wherein the said suspension is maintained for a period of about 4 to about 8 hours.

13. A crystalline form of Lacosamide characterized by data selected from a group consisting of a PXRD pattern having peaks at about 5.2, 6.7, 12.6, 16.2, 20.0 and 20.3±0.2 degrees 2-theta, a PXRD pattern as depicted in FIG. 5 and combinations thereof.

14. The crystalline form of claim 13, characterized by a PXRD pattern having peaks at about 5.2, 6.7, 12.6, 16.2, 20.0 and 20.3±0.2 degrees 2-theta.

15. The crystalline form of claim 13, characterized by a PXRD pattern as depicted in FIG. 5.

16. The crystalline form of claim 13, further characterized by a PXRD pattern having peaks at about 23.3 deg±0.2 degrees 2-theta.

17. A composition containing the crystalline form of Lacosamide of claim 13 and not more than about 10% by weight of a crystalline form of Lacosamide characterized by a PXRD pattern having peaks at about 8.3, 13.0, 16.6, 17.7 and 21.4 deg±0.2 degrees 2-theta.

18. A process for preparing a crystalline form of Lacosamide characterized by data selected from a group consisting of a PXRD pattern having peaks at about 5.2, 6.7, 12.6, 16.2, 20.0 and 20.3±0.2 degrees 2-theta, a PXRD pattern as depicted in FIG. 5 and combinations thereof, comprising providing a solution of Lacosamide in ethyl acetate and adding n-heptane to the obtained solution to obtain a suspension comprising the crystalline form.

19. The process of claim 18, wherein the solution is obtained by combining Lacosamide and ethyl acetate and heating the combination.

20. The process of claim 19, heating is done to a temperature of about 60° C. to about 70° C.

21. A process for preparing a crystalline form of Lacosamide characterized by data selected from a group consisting of a PXRD pattern having peaks at about 5.2, 6.7, 12.6, 16.2, 20.0 and 20.3±0.2 degrees 2-theta, a PXRD pattern as depicted in FIG. 5 and combinations thereof, comprising providing a solution of Lacosamide with ethyl acetate and cooling the solution to room temperature to obtain a suspension comprising the crystalline form.

22. The process of claim 21, wherein the solution is obtained by combining Lacosamide and ethyl acetate and heating the combination.

23. The process of claim 22, wherein heating is done to a temperature of about 60° C. to about 70° C.

24. The process of claims 21, wherein the cooling is done at a rate of about 1° C. per 1 minute.

25. Amorphous Lacosamide.

26. The amorphous Lacosamide of claim 25, characterized by a PXRD pattern as depicted in FIG. 9.

27. A process for preparing amorphous Lacosamide comprising lyophilization of an aqueous solution of Lacosamide.

28. A process for preparing amorphous Lacosamide comprising dissolving Lacosamide in t-butyl alcohol and removing the solvent under reduced pressure.

29. A pharmaceutical compositions comprising at least one of the polymorphic or amorphous forms of Lacosamide of claim 1, and at least one pharmaceutically acceptable excipient.

30. A pharmaceutical composition comprising at least one of the polymorphic or amorphous forms of Lacosamide prepared according to the processes of claim 6, and at least one pharmaceutically acceptable excipient.

31. A process for preparing a pharmaceutical composition comprising at least one of the polymorphic or amorphous forms of Lacosamide of claim 1, and at least one pharmaceutically acceptable excipient.

32. (canceled)

33. (canceled)

34. A method of treating a central nervous system disorder or alleviating pain comprising administering to a subject in need thereof of a pharmaceutical composition comprising at least one of the polymorphic or amorphous forms of Lacosamide of claim 1, and at least one pharmaceutically acceptable excipient.
